(19) United States
(12) Patent Application Publication
(10) Pub. No.: US 2013/0116539 A1
(43) Pub. Date: May 9, 2013

(54) OBJECT INFORMATION ACQUIRING APPARATUS AND CONTROL METHOD THEREOF

(71) Applicant: CANON KABUSHIKI KAISHA, Tokyo (JP)

(72) Inventor: Daisuke Nagao, Kawaguchi-shi (JP)

(73) Assignee: CANON KABUSHIKI KAISHA, Tokyo (JP)

(21) Appl. No.: 13/666,581

(22) Filed: Nov. 1, 2012

(30) Foreign Application Priority Data
Nov. 4, 2011 (JP) 2011-242272

(51) Int. Cl. A61B 6/00

(52) U.S. Cl. 600/407

(57) ABSTRACT

Provided is an object information acquiring apparatus including: a probe for receiving the acoustic waves; a holding member for holding the object; scanning unit for moving the probe in a first direction and a second direction; region designating unit for receiving information on a designated region which is a region for acquiring the characteristic information; and information processing unit for at least determining a scanning track in a scanning region to be scanned, wherein, of the scanning track in the scanning region when the first direction is used as a main scanning direction as a direction in which the probe moves while receiving the acoustic waves, and the scanning track when the second direction is used as the main scanning direction, the information processing unit uses the scanning track, in which scanning can be implemented in a shorter period of time.

### BACKGROUND OF THE INVENTION

1. Field of the Invention

The present invention relates to an object information acquiring apparatus and a control method thereof.

2. Description of the Related Art

Conventionally, numerous proposals have been made in relation to technology of using light for imaging a biological object image, and a photoacoustic imaging apparatus is one of such proposals. A photoacoustic imaging apparatus has shown particular usefulness in the diagnosis of skin cancer and breast cancer, and there are high expectations for its use as a medical device to replace the conventionally used ultrasonographs, X-ray devices, MRI devices and so on. When the tissue of the biological object is irradiated with the measuring light such as visible light or near-infrared light, the light absorbing substance inside the biological object, in particular substances such as hemoglobin in the blood, absorb the energy of the measuring light and instantaneously expand, and, consequently, acoustic waves are generated. This phenomenon is referred to as a photoacoustic effect, and the generated acoustic waves are also referred to as photoacoustic waves. With a photoacoustic imaging apparatus, information regarding the tissue of the biological object is visualized by measuring the photoacoustic waves. The technology of tomography using this kind of photoacoustic effect is also referred to as photoacoustic tomography (PAT). Based on this photoacoustic imaging technology, the light energy absorption density distribution; that is, the density distribution of the light absorbing substance in the biological object can be measured quantitatively and three-dimensionally.

Generally speaking, with breast cancer diagnosis in a breast oncology department, benign/malignant diagnosis is comprehensively performed via palpation or results upon using the plurality of modalities described above. Moreover, since the photoacoustic imaging apparatus can perform image diagnosis without radiation and non-invasively by using light for imaging the diagnostic image, there is a considerable advantage in terms of the burden on patients, and utilizing in the screening and early diagnosis of breast cancer is expected in substitute for an X-ray device which is difficult to use for diagnosis repeatedly.

With a photoacoustic imaging apparatus for performing breast cancer imaging, the light source and the acoustic probe are moved along the holding plate to perform scanning while the holding plates hold the object, and the three-dimensional photoacoustic wave image of the object is thereby obtained. The imaging time in the foregoing case includes the time up until acquiring the photoacoustic waves. Moreover, until the photoacoustic waves are acquired, it is necessary to restrain the object and impose burden thereon. In order to alleviate this burden, there are demands for imaging only the region that is required for the diagnosis. In order to meet the foregoing demands, there is technology which not only allows the user to designate the region to be imaged, but also allows the user to designate the required region in detail as a high resolution narrow region, designate a region where a normal image will suffice as a low resolution broad region, and synthesizing and displaying the low resolution image and the high resolution image. Consequently, the mutual positional relationship can be easily recognized visually. In addition, there is also technology which provides a position posture detector to obtain the position and posture of the acoustic probe during the scanning of a narrow region and the scanning of a broad region, and, even if the narrow region is changed, the high resolution image can be automatically synthesized with the low resolution image at the proper position (PTL1: Patent Literature 1).
PTL 1: Japanese Patent Application Laid-Open No. 2005-152346

### SUMMARY OF THE INVENTION

Nevertheless, with conventional technology, there was no unit for calculating a scanning track for efficiently scanning the imaging region (imaging designated region) designated by the user. Thus, there are demands for efficiently scanning and imaging the imaging designated region in the imaging performed by the photoacoustic imaging apparatus which acquires photoacoustic waves by causing the acoustic probe to perform scanning.

The present invention was devised in view of the foregoing problems, and an object of this invention is to provide technology capable of improving the scanning efficiency when the acoustic probe scans the imaging designated region during photoacoustic imaging.

The present invention provides an object information acquiring apparatus for acquiring characteristic information on an object by receiving acoustic waves propagated from the object,

the apparatus comprising:

a probe configured to receive the acoustic waves;

a holding member configured to hold the object;

scanning unit configured to move the probe along the holding member in a first direction and a second direction which intersects with the first direction;

region designating unit configured to receive information on a designated region which is a region for acquiring the characteristic information on the object; and

information processing unit configured to at least determine a scanning track in a scanning region to be scanned by the probe required for acquiring the characteristic information on the designated region,

wherein, of the scanning track in the scanning region when the first direction is used as amain scanning direction in which the probe moves while receiving the acoustic waves and the second direction is used as a sub scanning direction in which the probe does not receive the acoustic waves, and the scanning track in the scanning region when the second direction is used as the main scanning direction and the first direction is used as the sub scanning direction, the information processing unit uses the scanning track, in which scanning can be implemented in a shorter period of time, in the scanning region as the scanning track for the probe.

The present invention also provides a method of controlling an object information acquiring apparatus for acquiring characteristic information on an object by receiving, with a probe, acoustic waves propagated from an object held by a holding member,

the method comprising:

a scanning step in which scanning unit moves the probe along the holding member in a first direction and a second direction which intersects with the first direction;

a region designating step in which region designating unit receives information on a designated region which is a region for acquiring the characteristic information on the object; and

an information processing step in which information processing unit at least determines a scanning track in a scanning region to be scanned by the probe required for acquiring the characteristic information on the designated region,

wherein, in the information processing step, of the scanning track in the scanning region when the first direction is used as a main scanning direction in which the probe moves while receiving the acoustic waves and the second direction is used as a sub scanning direction in which the probe does not receive the acoustic waves, and the scanning track in the scanning region when the second direction is used as the main scanning direction and the first direction is used as the sub scanning direction, the scanning track, in which scanning can be implemented in a shorter period of time, in the scanning region is used as the scanning track of the probe.

According to the present invention, it is possible to provide technology capable of improving the scanning efficiency when the acoustic probe scans the imaging designated region during photoacoustic imaging.

Further features of the present invention will become apparent from the following description of exemplary embodiments with reference to the attached drawings.

### BRIEF DESCRIPTION OF THE DRAWINGS

FIG. 1 is a diagram showing the configuration of the photoacoustic wave imaging apparatus;

FIG. 2 is a diagram showing the scanning track of the probe when the X direction is the main scanning direction;

FIG. 3 is a flowchart showing the process of photoacoustic measuring in the imaging designated region;

FIG. 4 is a diagram showing the scanning track of the probe in the imaging designated region;

FIG. 5 is a diagram showing the scanning track of the probe when the Y direction is the main scanning direction;

FIG. 6A is a flowchart for calculating the scanning track;

FIG. 6B is a flowchart for calculating the scanning time Ta;

FIG. 6C is a flowchart for calculating the scanning time Tb; and

FIG. 7 is a diagram showing an example of the setting screen of the imaging designated region.

### DESCRIPTION OF THE EMBODIMENTS

The preferred embodiments of the present invention are now explained in detail with reference to the appended drawings. However, the scope of this invention is not limited to the illustrated examples. In the present invention, the acoustic waves include sound waves, ultrasound waves, photoacoustic waves, and elastic waves referred to as optical ultrasound waves, and the receiver (probe) receives the acoustic waves propagated in the object. The object information acquiring apparatus of the present invention includes apparatuses that use the photoacoustic effect of receiving acoustic waves generated in the object by irradiating light (electromagnetic waves) onto the object, and thereby acquiring the characteristic information in the object. In the case of apparatuses that use the photoacoustic effect, the acquired characteristic information in the object refers to object information which reflects the initial sound pressure of the acoustic waves that were generated based on irradiation of light, light energy absorption density that is derived from the initial sound pressure, absorption coefficient, concentration of substances configuring the tissue, and so on. The concentration of substances is, for example, oxygen saturation or oxy/deoxyhemoglobin concentration, or the like. Moreover, the characteristic information may also be acquired as distribution information showing the characteristics of the respective positions in the object rather than as numerical data. In other words, the distribution information of absorption coefficient distribution or oxygen saturation distribution may be acquired as the image data.

In the ensuing explanation, a photoacoustic imaging apparatus that uses the foregoing photoacoustic effect is explained as the representative example of an object information acquiring apparatus.

Note that the term “imaging” as used in the present invention refers to receiving acoustic waves from an object and acquiring the characteristic information on the object in the form of three-dimensional image data or the like. An imaging region is the region from which the characteristic information in the object is acquired, and is the region where three-dimensional images and the like are generated. In particular, the region that was designated by the user as the imaging region is also referred to as an imaging designated region.

Moreover, the object information acquiring apparatus of the present invention may also be an apparatus which uses the ultrasound echo technology of transmitting ultrasound waves to an object and receiving the reflected waves that were reflected inside the object, and thereby acquiring the characteristic information in the object. In the case of this apparatus that uses the ultrasound echo technology, the acquired characteristic information is information which reflects the difference in the acoustic impedance of the tissues inside the object.

### Embodiments

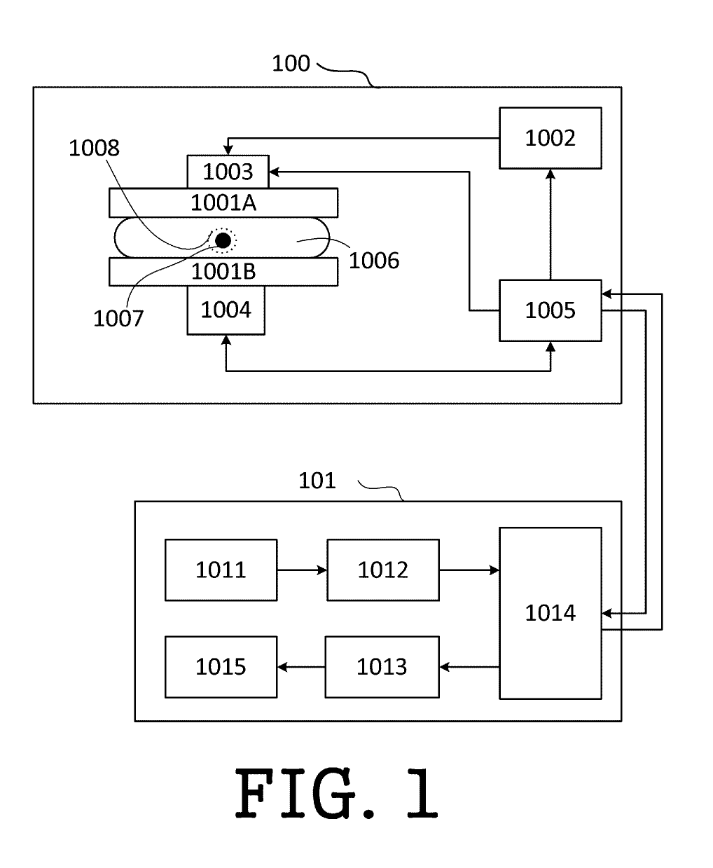

Embodiments that use the photoacoustic imaging apparatus of the present invention are now explained with reference to the drawings. As shown in FIG. 1, the photoacoustic imaging apparatus of this embodiment is configured from a photoacoustic wave signal measuring unit 100 and a photoacoustic wave information processing unit 101. The photoacoustic wave signal measuring unit 100 includes holding plates 1001A, 1001B for holding the object, a light source 1002 for generating measuring light, and an optical apparatus 1003 for causing the generated measuring light to be an intended mode. The photoacoustic wave signal measuring unit 100 additionally includes a detecting apparatus 1004 comprising an acoustic probe (hereinafter also referred to as a “probe”) for detecting the photoacoustic waves propagated from the object and which were generated based on the irradiation of light, and a measurement control unit 1005 for controlling the light source 1002, the optical apparatus 1003 and the detecting apparatus 1004.

Moreover, the photoacoustic wave information processing unit 101 includes an imaging region designating unit 1011 for receiving the designation of the imaging region from the user, a scanning track calculating unit 1012 for calculating the scanning track to be used for realizing efficient scanning, and an image generating unit 1013 for generating photoacoustic wave images from the imaging data. The photoacoustic wave information processing unit 101 further includes an information processing control unit 1014 for controlling the imaging region designating unit 1011, the scanning track calculating unit 1012 and the image generating unit 1013, and a display unit 1015 for displaying the photoacoustic wave image.

In FIG. 1, the object 1006 as the imaging target is fixed by the holding plates 1001 which compress and hold the object from either end thereof. The holding plate 1001 configuring the holding unit is configured by a pair of two holding plates 1001A and 1001B, and the holding position thereof is controlled by a holding mechanism not shown in order to change the holding interval and pressure. When there is no need to difference the holding plates 1001A and 1001B, they will be collectively indicated as the holding plates 1001. By sandwiching the object with the holding plates 1001 and thereby fixing the object to the apparatus, the measurement error resulting from the movement of the object 1006 can be reduced. Moreover, in accordance with the penetrating depth of the measuring light, the object 1006 can be adjusted to have a thickness that is suitable for photoacoustic measurement. Since the holding plates 1001 are positioned on the optical path of the measuring light, the holding plates 1001 need to have high transmittance relative to the measuring light, and at the same time the holding plates 1001 need to be made from a member having high acoustic compatibility with the probe as the measuring unit in the detecting apparatus 1004. For example, a member such as polymethylpentene, which is used in ultrasonographs or the like, is used. The holding plate corresponds to the holding member of the present invention.

The measuring light that is irradiated on the object 1006 is generated by the light source 1002. In this embodiment, the light source 1002 is configured from two light sources; namely, light source A and light source B not shown. The light source A and the light source B respectively generate light having different wavelengths. The light source 1002 generally uses a solid-state laser capable of emitting pulses having a central wavelength to the near-infrared region (for instance, Yttrium-Aluminium-Garnet laser or Titanium-Sapphire laser). The wavelength of the measuring light can be selected between 530 nm and 1300 nm according to the light absorbing substance (for instance, hemoglobin, glucose, cholesterol or the like) in the object 1006 to be imaged. For example, hemoglobin in the breast cancer neovascular vessels to be imaged generally absorbs light of 600 nm to 1000 nm. Meanwhile, a light absorber of water configuring the biological object becomes infinitesimal near 830 nm, the absorption of light relatively increased at 750 nm to 850 nm. Moreover, since the light absorption ratio changes depending on the hemoglobin state (oxygen saturation), there is a possibility that the functional change of the biological object may also be measured by comparing the foregoing changes.

Note that, in this embodiment, an example of using two light sources is illustrated, but it is also possible to use one light source or three or more light sources. Moreover, with alight source, the irradiation frequency is normally determined. This is set forth as a design value for continuously irradiating pulsed light of the intended intensity, but since the irradiation frequency affects the number of photoacoustic measurements to be communicated per unit time, the irradiation frequency is preferably as high as possible. In this embodiment, both the light source A and the light source B have an irradiation frequency of 20 Hz. Note that, in order to irradiate light of a plurality of wavelengths, it is also possible to use a wavelength variable laser rather than using a plurality of light sources. Moreover, the present invention is not limited to using light of a plurality of wavelengths, and can also be applied to cases where light of only one wavelength is used.

The optical apparatus 1003 for irradiating the measuring light, in an intended shape, from the light source 1002 to the object 1006 is configured from an optical system such as a lens, a mirror or an optical fiber, and a scanning mechanism for scanning the holding plates. Any optical system may be used so as long as it is possible to irradiate the measuring light, in an intended shape, from the light source 1002 to the object 1006.

When the object is irradiated with the measuring light generated by the light source 1002 via the optical apparatus 1003, the light absorber 1007 in the object absorbs the light, and emits photoacoustic waves 1008. Here, the light absorber 1007 corresponds to a sound source.

The detecting apparatus 1004 which receives the photoacoustic waves 1008 generated by the light absorber 1007 and converts such photoacoustic waves 1008 into an electric signal is configured from a probe, and a scanning mechanism for causing the probe to scan the holding plates. The probe is used for detecting the photoacoustic waves 1008 and converting the photoacoustic waves 1008 into an electric signal. The photoacoustic waves 1008 generated from the biological object are ultrasound waves of 100 KHz to 100 MHz. Thus, the detecting apparatus 1004 comprises a probe capable of receiving the foregoing frequency band. Any probe may be used so as long as the photoacoustic waves can be detected; for instance, a transducer using the piezoelectric phenomenon, a transducer using the resonance of light, or a transducer using the change in capacity. The probe of this embodiment is configured by a plurality of receiving elements being arrayed two-dimensionally. As a result of using this kind of two-dimensional arrayed elements, photoacoustic waves can be detected simultaneously at a plurality of locations, and it is thereby possible to shorten the detection time and reduce the influence of vibration or the like of the object. In particular, with this embodiment, let it be assumed that the receiving element pitch is 4 mm intervals in both the vertical and horizontal directions, and the receiving element array is 20 elements arrayed in both the vertical and horizontal directions. Moreover, in this embodiment, the object 1006 is irradiated with the measuring light at the front face of the probe. Thus, the optical apparatus 1003 and the detecting apparatus 1004 are arranged at positions facing each other with the object 1006 positioned therebetween. In addition, in order to maintain the foregoing positional relationship, scanning control which coordinates the optical apparatus 1003 and the detecting apparatus 1004 is performed.

The measurement control unit 1005 amplifies the electric signal of the photoacoustic waves obtained from the detecting apparatus 1004, and coverts the analog signal (analog electric signal) into a digital signal (digital electric signal). Moreover, estimation processing of the digital signals for noise reduction or control of the light source 1002, the optical apparatus 1003, and the detecting apparatus 1004 are performed. Moreover, the photoacoustic wave signal (digital signal after the estimation processing) is sent to an external device such as the information processing control unit 1014 from the measurement control unit 1005 via an interface not shown.

The estimation processing is performed for reducing the system noise and improving the S/N ratio of the photoacoustic wave signals by repeatedly measuring the same location of the object 1006 and performing averaging processing. A specific example is now explained. When certain l-number of receiving elements (hereinafter the first to l-th receiving elements) among the plurality of receiving elements (m-number of receiving elements) of the probe receive acoustic waves at the same scanning position relative to the object at different times, the electric signals output by the respective first to l-th receiving elements are estimated. Here, m and l are positive integers, and let it be assumed that l<m. The scanning width of the receiver is determined based the number of estimations (integration number) of the plurality of electric signals. The smaller the scanning width, the more superimposed the scanning regions will be, the integration number will thereby increase, and the S/N ratio will improve.

As the content of the control of the light source 1002, there is the selection of the light source A or the light source B, irradiation timing of the laser, and so on. As the control of the optical apparatus 1003 and the detecting apparatus 1004, there is the movement of the optical apparatus 1003 and the detecting apparatus 1004 to an appropriate position. While described in detail later, by two-dimensionally scanning the optical apparatus and the detecting apparatus relative to the object and measuring the same at the respective scanning positions, it is possible to acquire photoacoustic waves that are required for a broad imaging region even when a small probe is used. For example, it is possible to capture a photoacoustic wave image of the full breast in the imaging of the breast.

The photoacoustic wave information processing unit 101 generates and displays the photoacoustic wave image based on the photoacoustic wave signal received from the photoacoustic wave signal measuring unit 100, and calculates the efficient scanning track from the designated imaging region. As the photoacoustic wave information processing unit 101, an apparatus such as a personal computer or workstation comprising a sophisticated arithmetic processing function or graphic display function can be used.

The information processing control unit 1014 has an interface (not shown) that is equivalent to the signal measuring unit 100, and sends and receives imaging data and control orders of the photoacoustic wave information processing unit 101.

The image generating unit 1013 which generates photoacoustic wave image data by imaging the information of the optical characteristic distribution of the object based on the received photoacoustic wave signal can perform the following. Specifically, the image generating unit 1013 can apply various types of correction processing such as brightness adjustment or distortion correction to the generated photoacoustic wave image, and generate even more favorable information. The generated photoacoustic wave image is displayed on the display unit 1015.

The imaging region designating unit 1011 is an interface for receiving designations of the imaging region from the user through input means such as a mouse. The input means is not limited to a mouse or a keyboard, and may also be a pen tabulate type or a touch pad mounted on a display device surface. With respect to the designation of the imaging region, the imaging region can be designated based on the image captured with a camera (not shown) installed in a direction that is orthogonal to the holding plates that are compressing and holding the object.

The scanning track calculating unit 1012 will be described later in detail, but calculates the scanning track for efficiently scanning the designated imaging region. The scanning track that was calculated by the scanning track calculating unit 1013 is sent to the measurement control unit 1005 via the information processing control unit 1014.

With the photoacoustic imaging apparatus configured as described above, by performing imaging based on the photoacoustic effect, it is possible to image the optical characteristic distribution of the object and present a photoacoustic wave image. Note that, in FIG. 1, while the photoacoustic wave information processing unit is configured with separate hardware, the respective functions may also be consolidated to adopt an integrated configuration. Moreover, a part of the configuration; for instance, the display unit may be provided as outside hardware.

<Designation of Imaging Region and Scanning Region>

With respect to the designation of the imaging region, the imaging region can be designated based on the image captured by a camera (not shown) installed in a direction that is substantially orthogonal to the holding plates which are compressing and holding the object. When it is not possible to install the camera in a direction that is orthogonal to the holding plate, the image of the object may also be synthesized via image correction so that the image will be more visible to the user. The user, while referring to the camera image, designates the imaging region by anticipating that three-dimensional volume data, which includes up to the depth portion inside the object, will be acquired. The contents of the designation of the imaging region via the input means are received by the apparatus. The probe performs two-dimensional scanning along the holding plates so that the three-dimensional volume data of the imaging region can be acquired. This is the scanning region. Moreover, the designated region is converted from the coordinate system of the camera image into the scanning region of the apparatus coordinate system, and the probe is controlled so as to scan the corresponding position of the actual object.

### <Probe Scanning During Designation of Imaging Region>

The probe scanning when the imaging region is designated by the imaging region designating unit 1011 is now explained.

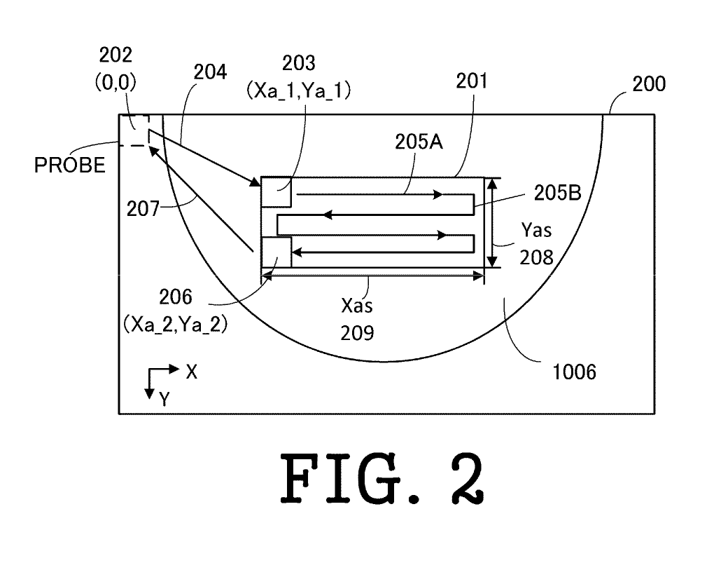

The conceptual diagram of FIG. 2 is the scanning track of the center of the probe when the imaging region is designated. Moreover, in the example shown the direction to be imaged is the main scanning direction, the direction that is not imaged is the sub scanning direction, and the X direction is used as the main scanning direction. The Y direction that intersects with the main scanning direction is the sub scanning direction. The X direction corresponds to the first direction of the present invention, and the Y direction corresponds to the second direction of the present invention.

The scannable region 200 represents the maximum region that can be scanned on the scanning surface, and the scanning designated region 201 represents the scanning region on the scanning surface corresponding to the designated imaging region. The probe performs scanning while moving (arrow 204) from the probe initial position 202 to the initial position 203 of the scanning designated region. Subsequently, all regions of the scanning designated region 201 are scanned in the main scanning direction 205A and the sub scanning direction 205B so as to perform photoacoustic wave measurement. Here, while the details will be explained later, depending on the designated imaging region, there are cases where the main scanning direction and the sub scanning direction are inverted. Subsequently, the probe moves (arrow 207) from the scanning end position 206 to the acoustic probe initial position 202.

### <Details of Scanning Track in Scanning Designated Region>

Details of probe scanning in the scanning designated region are now explained.

Here, in this embodiment, let it be assumed that the integration number of the electric signals per pixel is set to 40 times. In the case of this embodiment, since the number of elements of the probe is 20 elements in both the vertical and horizontal directions, and the integration number is 40 times, 20 estimations can be performed in a single outbound path by moving the probe one receiving element at a time (20 estimations can similarly be performed in a single return path).

Here, the region where the probe is moved in the main scanning direction and subject to photoacoustic measurement is defined as a stripe. In this embodiment, the size that the photoacoustic waves can be acquired with a single emission from the light source is the size of all element regions of the probe. In reality, while the region that is subject to photoacoustic wave measurement is a three-dimensional region including the depth direction, unless separately provided for herein, the two-dimensional projection plane on the scanning surface of the measurement system is indicated as a “stripe”.

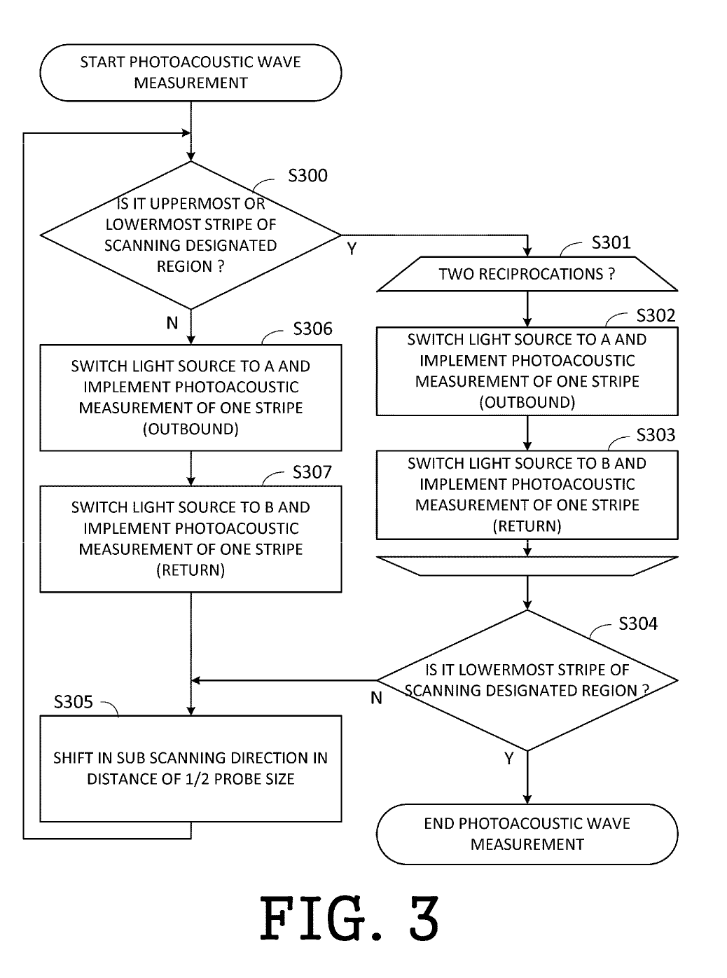

The probe scanning of the scanning designated region is now explained in detail with reference to the flowchart of FIG. 3 showing the flow of photoacoustic wave measurement.

The apparatus receives the input of the imaging region from the user and, after the probe moves to the scanning designated region initial position 203, photoacoustic wave measurement is started.

In step S300, whether the next measurement stripe is the uppermost stripe or the lowermost stripe of the scanning designated region; that is, whether the next measurement stripe is the first or last stripe to be measured is determined.

When the next measurement stripe is the first or last stripe to be measured (S300=Y), the probe reciprocates the target stripe twice (repeats steps S301 to S303 twice). In step S302, the light source is switched to the light source A and photoacoustic wave measurement for 1 stripe (outbound) is performed. In step S303, the light source is switched to the light source B and photoacoustic measurement of 1 stripe (return) is performed. Here, the reason why the probe makes two reciprocations is because, in this embodiment, the integration number is set to 40 times in both the light source A and the light source B, and therefore the integration number of one stripe will respectively be 20 times for a single outbound path and a single return path.

Next, in step S304, whether the stripe that was just measured is the lowermost stripe of the scanning designated region is determined. When the measured stripe is the lowermost stripe (S304=Y), the photoacoustic wave measurement of the imaging designated region is ended. When the measured stripe is not the lowermost stripe (S304=N); that is, when the measured stripe is the uppermost stripe, the routine proceeds to step S305, and the probe is moved in the sub scanning direction in a distance which is half the probe size. Here, the reason why the probe is moved in a distance which is half the probe size is to cause the probe to measure various receiving elements as much as possible in all pixels of the imaging designated region. In this embodiment, while the movement distance in the sub scanning direction was half the probe size, for instance, the probe may also be moved the same distance as the probe size or a distance that is ⅔ of the probe size, and so on.

Meanwhile, after this flow is started, when the measured stripe is not the uppermost stripe or the lowermost stripe of the scanning designated region (S300=N), the routine proceeds to step S306. In addition, the light source is switched to the light source A, and photoacoustic wave measurement of 1 stripe (outbound) is performed (step S306). Subsequently, the light source is switched to the light source B, and photoacoustic wave measurement of 1 stripe (return) is performed (step S307). Subsequently, the probe is moved in the sub scanning direction in a distance that is half the probe size (step S305). Here, since the probe is moved in a distance of half the probe size for each stripe, excluding the uppermost or the lowermost stripe, the integration number of both the light source A and the light source B will reach 40 times in a single reciprocation.

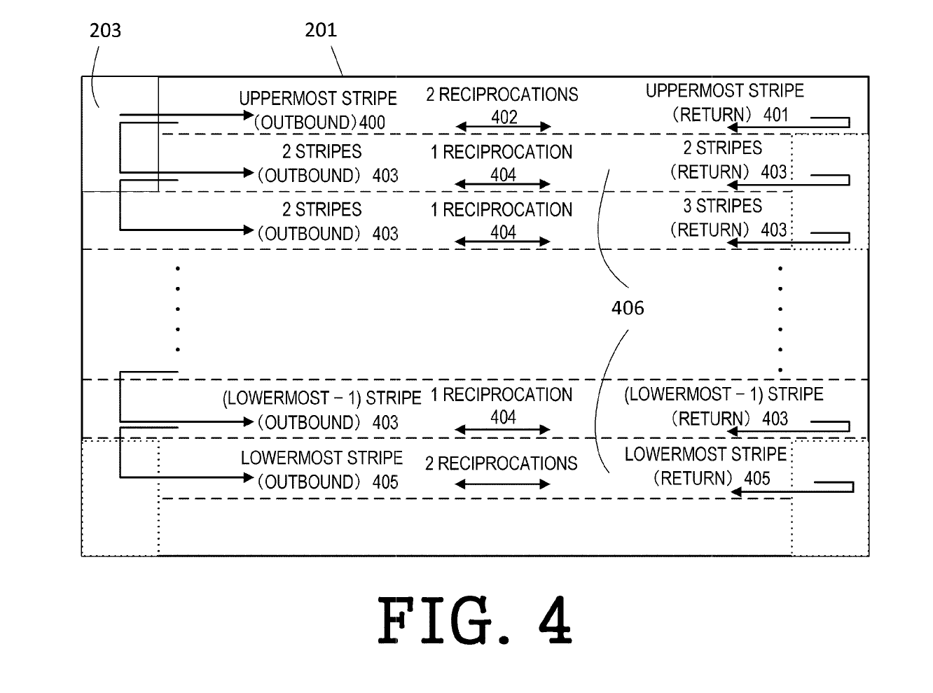

The foregoing scanning track is now conceptually explained in detail with reference to FIG. 4. The photoacoustic wave signal is measured with the light source A in the outbound path of the uppermost stripe 400 and the photoacoustic wave signal is measured with the light source B in the return path of the uppermost stripe 401 from the scanning designated region initial position 203. Note that the actual height of the stripe coincides with the height of the probe initial position 203; that is, the size of the probe in the Y direction, but since there is an overlapping portion in the adjacent stripes in the sub scanning direction of FIG. 4, the stripe that was scanned later is shown with respect to such portion.

Subsequently, the second reciprocation is performed (arrow 402). Next, the photoacoustic wave signal is measured with the light source A in the outbound path and with the light source B in the return path from the second stripe to the (lowermost−1) stripe (403) for a single reciprocation (arrow 404). In the lowermost stripe 405, the same scanning as the foregoing uppermost stripe is performed. Here, while the integration number will exceed 40 times at least with the upper half stripe of the uppermost stripe or the lower half stripe of the lowermost stripe, there is no problem since this will lead to the improvement in the S/N ratio. 406 is a region where the integration number exceeds 40 times.

This embodiment is based on the premise of the foregoing apparatus configuration and apparatus that performs the foregoing probe scanning.

Calculation of the actual scanning track is now explained in detail.

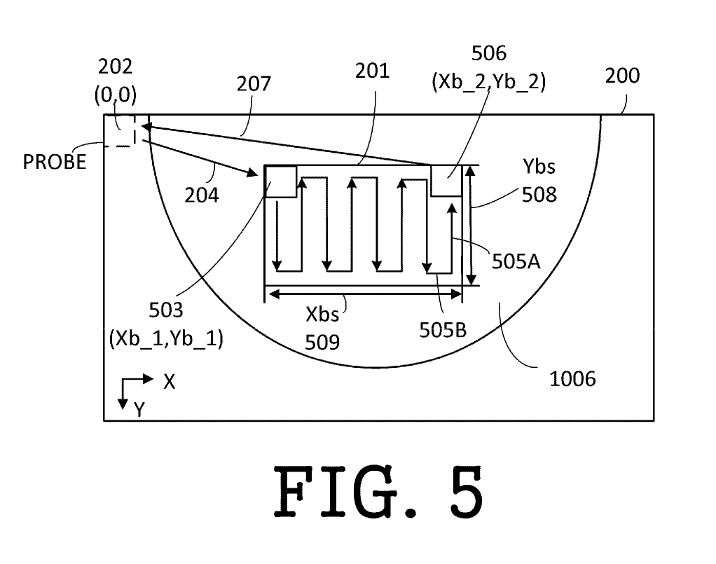

FIG. 5 is the scanning track of the center of the probe when the Y direction is the main scanning direction in comparison to FIG. 2.

With the scanning track of this embodiment, the scanning time when the X direction is the main scanning direction as shown in FIG. 2 and the scanning time when the Y direction is the main scanning direction as shown in FIG. 5 are compared as described later, and the shorter scanning time is adopted. However, the scanning track is not limited to the track that is illustrated in this embodiment and, for example, a track of a whirlpool shape (spiral shape) may also be used.

### <Explanation of Photoacoustic Wave Acquisition Time, Restraining Time and Scanning Time>

In order to perform accurate imaging with the photoacoustic imaging apparatus, the test subject needs to be restrained such as by fixing a part of the test subject's body. Particularly when imaging the breast of the test subject, the test subject is restrained in a manner where the breast is compressed and fixed. Since the restraint of the test subject often involves distress, the shortening of the time from the start of measurement to the release of the test subject will be advantageous for the test subject, as well as for the physicians and operators performing the imaging. This kind of time is referred to as the restraining time, and the explanation is continued below.

While the calculation method of the imaging time; that is, the restraining time, will differ depending on whether the test subject is restrained after measurement is started or restrained before measurement is started, the calculation of the restraining time in the present invention may adopt either method. In other words, when performing restraint processing of automatically compressing and fixing the test subject's breast after measurement is started, the time calculated by adding the time required for acquiring the photoacoustic waves to the restraint processing time and the release processing time shall be the restraining time of the test subject. When the compression and fixation of the test subject's breast are completed with the procedures and manual apparatus scanning by the physician or operator before measurement is started, the time calculated by adding the time required for acquiring the photoacoustic waves to the release processing time shall be the restraining time of the test subject. In this embodiment, the latter is taken as an example and explained.

Moreover, with respect to the time required for acquiring the photoacoustic waves, the time that the probe is moving along the holding plate (hereinafter referred to as the “scanning time”) accounts for much of such time. Thus, the restraining time basically corresponds to the time obtained by adding the scanning time and the time required for releasing the test subject. Here, the time required for releasing the test subject is generally the same regardless of the scanning track. Accordingly, in this embodiment, the time that is used for calculating the scanning track shall only be the scanning time. In other words, by calculating the scanning track so that the scanning time is shortened, the restraining time of the test subject can be shortened.

The scanning time is configured from (1) to (4) below.

(1) Scanning designated region initial position moving time Ta1 (or Tb1) which is the time required for the simple movement from the probe initial position 202 to the scanning designated region initial position 203 (or 503).

(2) Main scanning direction moving time Ta2 (or Tb2) which is the time required for moving while acquiring the photoacoustic waves of the scanning designated region in the main scanning direction 205A (or 505A).

(3) Sub scanning direction moving time Ta3 (or Tb3) which is the time required for the simple movement of the scanning designated region in the sub scanning direction 205B (or 505B).

(4) Probe initial position moving time Ta4 (or Tb4) which is the time required for the simple movement from the scanning end position 206 (or 506) to the probe initial position 202.

### <Calculation of Scanning Track>

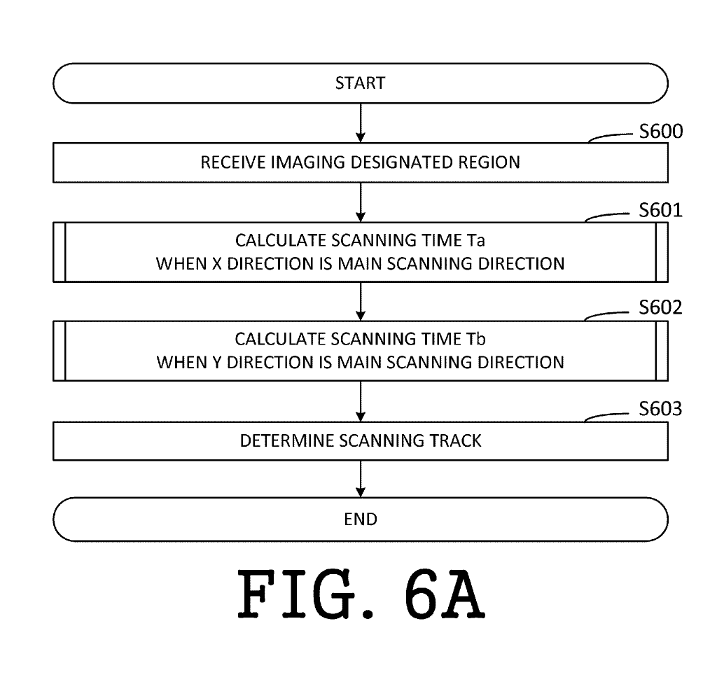

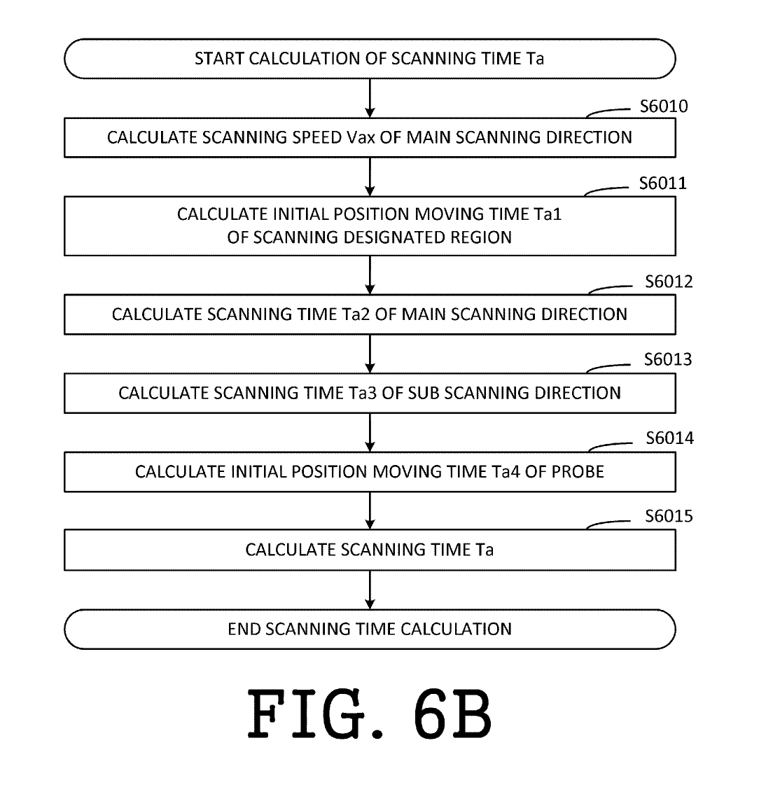

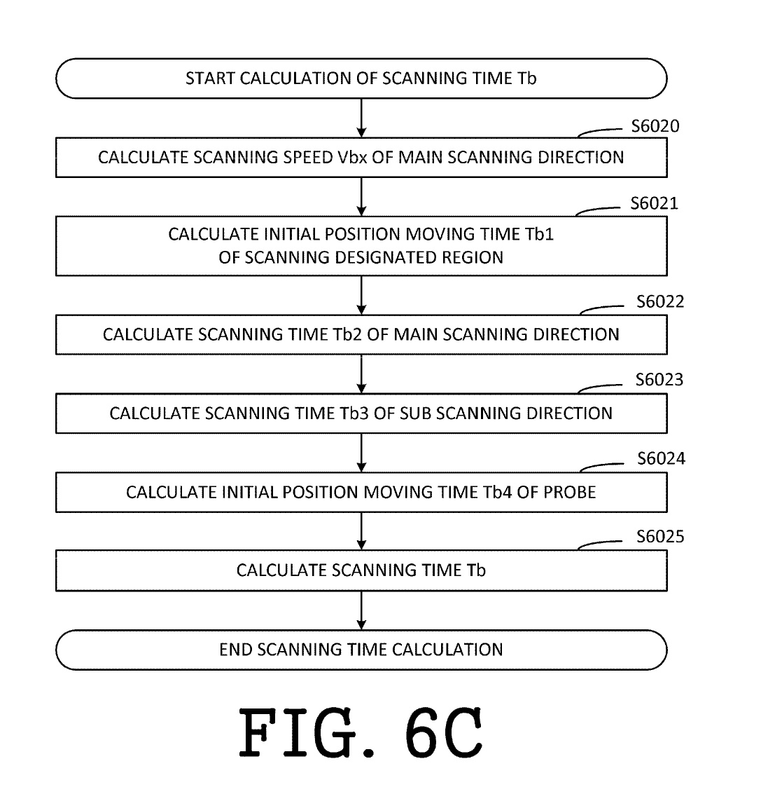

Calculation of the scanning track is now explained with reference to the flowchart of FIG. 6. FIG. 6A shows the flow of the overall calculation processing. The detailed processing of step S601 in FIG. 6 is shown in FIG. 6B, and the detailed processing of step S602 is shown in FIG. 6C.

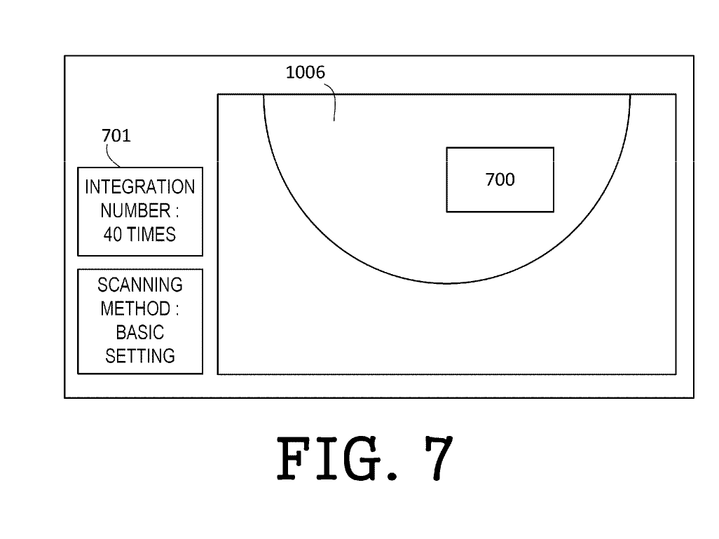

In step S600, the apparatus receives the imaging region that was designated by the user via the imaging region designating unit 1011. An example of designating the imaging region is shown in FIG. 7. 700 represents the imaging designated region designated by the user. Here, the measuring conditions of the photoacoustic measurement may also be set. For example, the integration number can be set in the integration number setting window 701.

In step S601, the scanning time Ta when the X direction is the main scanning direction as shown in FIG. 2 is calculated. Details of this processing are now explained with reference to the flowchart of FIG. 6B.

In step S6010, the scanning speed of the main scanning direction is calculated. Let it be assumed that the number of elements of the acoustic probe in the X direction is Enxa (elements), the element pitch is Epitcha (mm), the integration number of photoacoustic measurement is Mn (times), the movement distance in the sub scanning direction per integration is ½ the probe size, the number of laser light sources is 2 (light sources), and the light-emitting frequency of the laser light source is LHz (Hz). In order to simplify the explanation, let it be assumed that the integration number Mn is an integration of the number of elements Enxa. Here, the scanning speed Vax (mm/sec) of the acoustic probe and the laser light source in the main scanning direction is calculated with the following Formula (1), and the number of scans San (scans) is calculated with the following Formula (2).

Vax=Epitcha×LHz (1)

San=(Mn/Enxa)×2×(½) (2)

In the case of this embodiment, as described above, since the number of elements of the probe is 20 elements (Enxa=20) in the X direction, and the integration number is 40 times (Mn

=40), based on Formula (2) the number of scans San=2. Thus, by moving the acoustic probe 104 one receiving element at a time, 40 estimations can be performed in a single reciprocation.

Moreover, since the single reciprocation element pitch is 4 mm (Epitcha=4), and the light-emitting frequency of the laser light source is 20 Hz (LHz=20), based on Formula (1), the scanning speed Vax of the measurement system upon measurement will be 80 mm/sec.

The scanning speed for measurement obtained as described above is used for calculating the measurement time explained later.

Under more complex conditions, such as when the integration number is smaller than the number of elements Enxa in the main scanning direction, or a multiple of a value that is smaller than Enxa, the integration number per reciprocal movement of the probe will decrease. In the foregoing case, since scanning can be performed with the displacement of the probe being a shift of two or more pixels per unit time, the setting of the scanning speed will be high. The moving speed of the probe is not limited to the method illustrated in this embodiment, and various algorithms may be applied for adjusting the scanning speed while depending on the measurement conditions or apparatus configuration.

Since the scanning speed calculation function in this embodiment aims to obtain the prove moving speed for photoacoustic measurement, the reference parameters and algorithms are not limited to the methods described in this embodiment.

In step S6011, the scanning region initial position moving time Ta1 is calculated. The coordinates of the probe initial position 202 are set as (0,0), the coordinates of the scanning designated region initial position 203 are set as (Xa_1, Ya_1), and the scanning speed of the probe other than during photoacoustic measurement is set as Vxy (mm/sec). Here, the scanning region initial position moving time Ta1 is represented with the following Formula (3).

$$\begin{matrix}[ Math . 1 ] & \\ Ta 1 = \frac{\sqrt{Xa\_ {1}^{2} + Ya\_ {1}^{2}}}{Vxy} & ( 3 )\end{matrix}$$

In step S6012, the main scanning direction moving time Ta2 is calculated. Here, the number of stripes Na that covers the scanning designated region when the movement distance of the sub scanning direction is set to be ½ of the probe size is represented with the following Formula (4). Note that Yas is the length 208 of the scanning designated region in the Y direction, Enxb is the number of elements of the acoustic probe in the Y direction, and Epitchb is the element pitch. The Na obtained here represents the number of movements made by the probe from end to end in the main scanning direction of the scanning designated region.

$$\begin{matrix}[ Math . 2 ] & \\ Na = ceil ( \frac{Yas}{\frac{Enxb \times Epitchb}{2}} ) & ( 4 )\end{matrix}$$

Thus, the total movement distance of the scanning designated region in the main scanning direction will be Xas×(Na+2)×San when the length 209 of the scanning designated region in the X direction is Xas. Accordingly, the main scanning direction time Ta2 is represented with the following Formula (5).

$$\begin{matrix}[ Math . 3 ] & \\ Ta 2 = \frac{Xas \times ( Na + 1 ) \times San}{Vax} & ( 5 )\end{matrix}$$

In step S6013, the sub scanning direction time Ta3 is calculated. The sub scanning direction moving time Ta3 is represented with the following Formula (6).

Ta3=Yas/Vxy (6)

In step S6014, the probe initial position moving time Ta4 is calculated. When the scanning end position 206 is (Xa_2,Ya_2), the probe initial position moving time Ta4 is represented with the following Formula (7).

$$\begin{matrix}[ Math . 4 ] & \\ Ta 4 = \frac{\sqrt{Xa\_ {2}^{2} + Ya\_ {2}^{2}}}{Vxy} & ( 7 )\end{matrix}$$

In step S6015, the scanning time Ta is calculated. The scanning time Ta is represented with the following Formula (8).

Ta=Ta1+Ta2+Ta3+Ta4 (8)

Here, the scanning speed in the scanning time calculation was calculated as being constant in all cases, but a more strict scanning speed giving consideration to the initial acceleration or the like may also be used. The scanning speed used in the scanning time calculation is not limited to the method described in this embodiment.

In step S602, the scanning time Tb when the Y direction is the main scanning direction as shown in FIG. 5 is calculated. This processing shown in the flowchart of FIG. 6C is basically the same as the processing of FIG. 6B.

Here, the respective parameters shall be as follows. Moreover, the number of stripes Nb is represented with Formula (9).

Number of elements of the acoustic probe in the Y direction: Enxb
Element pitch: Epitchb (mm)
Number of scans: Sbn (times)
Sbn=(Mn/Enxb)×2×(½)
Scanning designated region initial position 503: (Xb_1, Yb_1)
Length 208b of the scanning designated region in the Y direction: Ybs
Length 209b of the scanning designated region in the X direction: Xbs
Number of stripes: Nb

$$\begin{matrix}[ Math . 5 ] & \\ Nb = ceil ( \frac{Xbs}{\frac{Enxa \times Epitcha}{2}} ) & ( 9 )\end{matrix}$$

Total movement distance of the scanning designated region in the main scanning direction: Ybs×(Nb+1)×Sbn
Scanning end position 506: (Xb_2,Yb_2)

Consequently, the scanning speed Vbx (mm/sec), Tb1, Tb2, Tb3, Tb4, Tb of the main scanning direction calculated in step S6020 to step S6025 can be calculated with the following Formula (10) to Formula (15) as with step S6010 to step S6015.

Vbx=Epitchb×LHz (10)

$$\begin{matrix}[ Math . 6 ] & \\ Tb 1 = \frac{\sqrt{Xb\_ {1}^{2} + Yb\_ {1}^{2}}}{Vxy} & ( 11 ) \\ Tb 2 = \frac{Ybs \times ( Nb + 1 ) \times Sbn}{Vbx} & ( 12 ) \\ Tb 3 = \frac{Xbs}{Vxy} & ( 13 ) \\ Tb 4 = \frac{\sqrt{Xb\_ {2}^{2} + Yb\_ {2}^{2}}}{Vxy} & ( 14 )\end{matrix}$$

Tb=Tb1+Tb2+Tb3+Tb4 (15)

In step S603, the scanning track is determined. The calculated Ta and Tb are compared and the scanning track, in which scanning can be implemented in a shorter period of time, is selected. When Ta and Tb are the same, a scanning track with either the X direction or the Y direction as the main scanning direction may be selected.

In this embodiment, as shown in Formula (8) and Formula (15), in addition to the moving time in the scanning designated region, the scanning time was calculated by including the scanning designated region initial position moving time Ta1 (or Tb1) and the probe initial position moving time Ta4 (or Tb4). In addition, the scanning track was determined so as to shorten the scanning time. Nevertheless, in the present invention, it is also possible to determine the scanning track without giving consideration to the scanning designated region initial position moving time Ta1 (or Tb1) and the probe initial position moving time Ta4 (or Tb4). In other words, the scanning time may be determined with only the main scanning direction moving time Ta2 (or Tb2) and the sub scanning direction moving time Ta3 (or Tb3) in the scanning designated region as the scanning time. In other words, in the present invention, it will suffice so as long as the scanning track which shortens the scanning time is determined as the scanning track of the probe by giving consideration at least to the scanning time with respect to the scanning designated region.

The apparatus of this embodiment is an apparatus which calculates the corresponding scanning region based on the imaging designated region designated by the user, determines the efficient scanning track, and thereby performs photoacoustic imaging. By streamlining the scanning track, the time that the test subject is restrained can be reduced, and the burden on the test subject can be alleviated.

The effects of this embodiment are now explained with reference to specific examples.

In the first specific example, the respective parameters are as follows. The scanning region size is as follows; namely, X direction length Xas=100 mm, and Y direction length Yas=40 mm. Coordinates of the scanning designated region initial position are (Xa_1,Ya_1)=(50, 70) mm. The number of elements is 20 (Enxa=20) in the X direction and 20 (Enxb=20) in the Y direction. The element pitch is 1 mm (Epitcha=1) in the X direction, and 1 mm (Epitchb=1) in the Y direction. The integration number is 40 times. The movement distance to the sub scanning direction is ½ of the probe size. The number of light sources is 2. The light-emitting frequency of the light source is LHz=2 Hz. The scanning speed of the probe other than during photoacoustic measurement is Vxy=50 (mm/sec).

Here, based on Formula (1) to Formula (8), the scanning time when the X direction is the main scanning direction will be Ta=504.9 sec. Meanwhile, based on Formula (9) to Formula (15), the scanning time when the Y direction is the main scanning direction will be Tb=565.7 sec. Accordingly, by applying the present invention, measurement in the X direction as usual may be selected. Consequently, it is possible to shorten the time required for the measurement, and thereby alleviate the burden on the test subject.

In the second specific example, the respective parameters are as follows. The scanning region size is as follows; namely, X direction length Xas=100 mm, and Y direction length Yas=41 mm. The other conditions are the same as the first specific example.

Here, based on Formula (1) to Formula (8), the scanning time when the X direction is the main scanning direction will be Ta=605.0 sec. Meanwhile, based on Formula (9) to Formula (15), the scanning time when the Y direction is the main scanning direction will be Tb=572.7 sec. Thus, by applying the present invention, measurement with the Y direction as the scanning direction, rather than the usual X direction, may be selected. Consequently, it is possible to shorten the time required for the measurement, and thereby alleviate the burden on the test subject.

In the third specific example, the respective parameters are as follows. The scanning region size is as follows; namely, X direction length Xas=100 mm, and Y direction length Yas=50 mm. Coordinates of the scanning designated region initial position are (Xa_1,Ya_1)=(50, 65) mm. The number of elements is 20 (Enxa=20) in the X direction, and 10 (Enxb=10) in the Y direction. The other conditions are the same as the first specific example.

Here, based on Formula (1) to Formula (8), the scanning time when the X direction is the main scanning direction will be Ta=1105.1 sec. Meanwhile, based on Formula (9) to Formula (15), the scanning time when the Y direction is the main scanning direction will be Tb=985.8 sec. Thus, by applying the present invention, measurement with the Y direction as the scanning direction, rather than the usual X direction, may be selected. Consequently, it is possible to shorten the time required for the measurement, and thereby alleviate the burden on the test subject.

The preferred embodiments of the present invention were explained above, but the present invention is not limited to such embodiments, and may be variously modified and changed within the scope of the gist of this invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-242272, filed on Nov. 4, 2011, which is hereby incorporated by reference herein its entirety.

What is claimed is:

1. An object information acquiring apparatus for acquiring characteristic information on an object by receiving acoustic waves propagated from the object,
the apparatus comprising:
a probe configured to receive the acoustic waves;
a holding member configured to hold the object;
scanning unit configured to move the probe along the holding member in a first direction and a second direction which intersects with the first direction;
region designating unit configured to receive information on a designated region which is a region for acquiring the characteristic information on the object; and
information processing unit configured to at least determine a scanning track in a scanning region to be scanned by the probe required for acquiring the characteristic information on the designated region,
wherein, of the scanning track in the scanning region when the first direction is used as a main scanning direction in which the probe moves while receiving the acoustic waves and the second direction is used as a sub scanning direction in which the probe does not receive the acoustic waves, and the scanning track in the scanning region when the second direction is used as the main scanning direction and the first direction is used as the sub scanning direction, the information processing unit uses the scanning track, in which scanning can be implemented in a shorter period of time, in the scanning region as the scanning track for the probe.

2. The object information acquiring apparatus according to claim 1, wherein
the probe includes m-number of receiving elements (m is a positive integer) configured to receive the acoustic waves and respectively convert the acoustic waves into electric signals, and
the information processing unit calculates a length of scanning time with respect to the scanning region by using a integration number of integration of integrating a plurality of electric signals which are respectively output from certain l-number of receiving elements (l is a positive integer and l<m) among the m-number of receiving elements when the l-number of receiving elements receive the acoustic waves at the same scanning position in the object.

3. The object information acquiring apparatus according to claim 2,
wherein the acoustic waves propagated from the object are photoacoustic waves that are generated from the object irradiated with light.

4. The object information acquiring apparatus according to claim 3,
wherein the information processing unit:
calculates a scanning speed in the main scanning direction and a scanning speed in the sub scanning direction by using an element pitch of the receiving elements and the integration number and an irradiation frequency of the light, and
calculates a scanning time for the scanning region based on the calculated scanning speed in the main scanning direction and the sub scanning direction.

5. The object information acquiring apparatus according to claim 1,
wherein the region designating unit displays an image of the object acquired by a camera to a user, and receives an input of the designated region from the user who has viewed the image of the object.

6. The object information acquiring apparatus according to claim 5,
wherein the information processing unit calculates the scanning time on the basis of a time that is required for the probe to move while receiving the acoustic waves in the main scanning direction, a time that is required for the probe to move in the sub scanning direction, a time that is required for the probe to move from an initial position to the scanning region when acquisition of the characteristic information is started, and a time that is required for the probe to move to the initial position after acquisition of the characteristic information is complete.

7. The object information acquiring apparatus according to claim 1,
wherein, when a region, in which the probe moves upon receiving the acoustic waves in the main scanning direction, is a stripe, a movement distance for the probe in the sub scanning direction is smaller than a width of the stripe.

8. The object information acquiring apparatus according to claim 2,
wherein the acoustic waves propagated from the object are acoustic waves that have been reflected inside the object among the acoustic waves transmitted to the object.

9. A method of controlling an object information acquiring apparatus for acquiring characteristic information on an object by receiving, with a probe, acoustic waves propagated from an object held by a holding member,
the method comprising:
a scanning step in which scanning unit moves the probe along the holding member in a first direction and a second direction which intersects with the first direction;
a region designating step in which region designating unit receives information on a designated region which is a region for acquiring the characteristic information on the object; and
an information processing step in which information processing unit at least determines a scanning track in a scanning region to be scanned by the probe required for acquiring the characteristic information on the designated region,
wherein, in the information processing step, of the scanning track in the scanning region when the first direction is used as a main scanning direction in which the probe moves while receiving the acoustic waves and the second direction is used as a sub scanning direction in which the probe does not receive the acoustic waves, and the scanning track in the scanning region when the second direction is used as the main scanning direction and the first direction is used as the sub scanning direction, the scanning track, in which scanning can be implemented in a shorter period of time, in the scanning region is used as the scanning track of the probe.
